(19) United States
(12) Patent Application Publication
(10) Pub. No.: US 2025/0195348 A1
(43) Pub. Date: Jun. 19, 2025

(54) DOPED TITANIUM DIOXIDE COMPOSITIONS AND METHODS OF USE IN WHITENING TEETH

(71) Applicants: THE BOARD OF REGENTS OF THE UNIVERSITY OF OKLAHOMA, Norman, OK (US); UNIVERSIDADE ESTADUAL DE CAMPINAS - UNICAMP, São Paulo (BR)

(72) Inventors: Fernando L. ESTEBAN FLOREZ, Oklahoma City, OK (US); Sharukh S. KHAJOTIA, Edmond, OK (US); Matheus KURY RODRIGUES, Tramandai (BR)

(73) Assignees: THE BOARD OF REGENTS OF THE UNIVERSITY OF OKLAHOMA, Norman, OK (US); UNIVERSIDADE ESTADUAL DE CAMPINAS - UNICAMP, São Paulo (BR)

(21) Appl. No.: 18/847,340

(22) PCT Filed: Mar. 17, 2023
(86) PCT No.: PCT/US2023/015428

§ 371 Date: Sep. 16, 2024

Related U.S. Application Data
(60) Provisional application No. 63/321,158, filed on Mar. 18, 2022.

(51) Int. Cl. A61K 8/22; A61K 8/02; A61K 8/04; A61K 8/29; A61Q 11/00

(52) U.S. Cl. CPC A61K 8/22; A61K 8/0241; A61K 8/042; A61K 8/29; A61Q 11/00; A61K 2800/413

(57) ABSTRACT

A dental composition, and methods of use thereof, for causing tooth whitening and/or tooth mineralization without causing tooth hypersentivity. The dental composition contains a gel-forming thickening agent and nitrogen-doped titanium dioxide nanoparticles. The nitrogen-doped titanium dioxide nanoparticles may be co-doped with fluorine and/or silver. The dental composition may contain a bleaching agent.

### BACKGROUND

In-office power bleaching (IPB) is considered an ultraconservative and minimally invasive treatment capable of resolving dental discolorations (low to moderate) in as short as one clinical session. The IPB treatment typically involves three clinical sessions (e.g., 45 min/each; 7-day apart) where hydrogen peroxide (HP)-containing bleaching gels (HP, 35% to 45%) are used in combination with visible light irradiation to promote the attainment of immediate esthetic outcomes. IPB's underlying mechanism of action revolves around the generation of reactive oxygen species (ROS). Upon generation, these short-lived and highly-reactive free radicals must be efficiently transported from the gel to the dentin-enamel junction (DEJ). Once at the DEJ, free radicals will then break conjugated double bonds present in large organic molecules (chromophores) through a non-specific oxidative process.

Even though several reports have demonstrated the bleaching efficacy of IPB, other studies have indicated that the utilization of these highly caustic bleaching agents may result in the occurrence of adverse effects (short- and long-term) including irreversible changes in enamel's topography and chemical make-up, decreased surface microhardness, increased surface roughness, diminished bond strength and reduced fracture resistance. From the clinical standpoint, the most prevalent adverse effect reported by patients and clinicians is mild to severe dentin hypersensitivity (DH). According to previous studies, there is a strong and positive correlation among dentin hypersensitivity, HP concentration and pulpal cytotoxicity, where the higher the HP concentration, the stronger the dentin hypersensitivity and more durable are the effects.

In this critical context, several research groups have tried to overcome limitations described by adding ions of calcium or fluorine in the formulation of highly concentrated bleaching gels. Even though results reported have demonstrated that adverse effects, such as diminished enamel microhardness and rougher surfaces were less pronounced with the utilization of calcium- or fluorine-containing gels, subsequent studies have shown that promising results initially reported, were limited to the outermost layers of enamel, and did not prevent the loss of minerals at subsurface levels, thereby restricting the therapeutic effect of novel formulations proposed. Follow-up studies investigated the efficacy of experimental protocols modulated by low-concentrated bleaching gels (6-15%) and near-UVA wavelengths (405±15 nm), as an alternative approach to reduce the incidence of dentin hypersensitivity while trying to achieve desirable whitening outcomes. Even though the utilization of low-concentrated bleaching gels resulted in lower incidences of DH, the bleaching efficacies reported (in terms of ΔE and whitening index [WI]) were considered poor because outcomes were much less intense and durable, as compared to those attained with gels containing high HP concentrations.

Recent approaches focused on the incorporation of metal oxides, such as titanium dioxide (TiO₂, P25 Degussa) and nitrogen-doped titanium dioxide (N_TiO₂) nanoparticles (NPs), into the formulation of commercially-available bleaching gels containing high HP concentrations. In theory, the incorporation of these semiconductors would improve the dissociation of HP into ROS, by a photo-physical process, where photons are converted into thermal energy. However, despite the theorical feasibility of the process, experimental bleaching gels containing varying concentrations of metal oxide nanoparticles were demonstrated to be clinically ineffective when compared to unaltered gels containing HP (either 15% or 35%).

Successful fabrication of N_TiO₂ (6-15 nm) using highly controllable, reproducible and green solvothermal reactions has recently been reported (Esteban Florez, F. L. et al. Antibacterial dental adhesive resins containing nitrogen-doped titanium dioxide nanoparticles. Mater Sci Eng C Mater Biol Appl 93, 931-943 (2018)). In that study, nanoparticles synthesized were incorporated into commercially-available dental adhesive resins (OptiBond Solo Plus, Kerr Corp.; OPTB) with the objective of imparting non-leaching antibacterial and biomimetic functionalities to the parental polymer. This synthesis route results in the attainment of pure and crystalline TiO₂ nanoparticles (anatase phase) that are electron deficient, display high levels of nitrogen doping, have well-defined pore structure, large surface areas, facilitate the generation of electron-hole pairs, and are capable of efficiently absorbing visible wavelengths (400 to 700 nm) while generating significant amounts of perhydroxyl (HO₂^{•}) and hydroxyl (OH^{•}) radicals, which are long-lived species of oxygen. Further work demonstrated the successful solvothermal synthesis of TiO₂ nanoparticles that were co-doped with either nitrogen and fluorine (NF_TiO₂) or nitrogen and silver (NAg_TiO₂), their functionalization into OPTB, and the testing of antibacterial properties (in dark and light irradiated conditions) against Streptococcus mutans using a newly developed and optimized high throughput bioluminescence assay (Esteban Florez, F. L. et al. Optimization of a real-time high-throughput assay for assessment of Streptococcus mutans metabolism and screening of antibacterial dental adhesives. Dent Mater 36, 353-365 (2020); Esteban Florez, F. L. et al. Advanced characterization of surface-modified nanoparticles and nanofilled antibacterial dental adhesive resins. Scientific Reports 10, 9811 (2020)). According to results reported, experimental materials containing 30% of either NF_TiO₂ or NAg_TiO₂ displayed antibacterial behaviors that were comparable to those attained with Clearfil SE Protect (Kuraray Co.; fluoride-releasing material) independently of light irradiation conditions. These findings have not only indicated that the nanotechnology reported has a strong potential to be translated into commercial products capable of sustaining long-term antibacterial properties, but the promising antibacterial effects observed in the absence of light, corroborate findings that nanoparticles synthesized through solvothermal processes are capable of generating long-lived species of oxygen. Even though the efficacy of IPB has been previously demonstrated by numerous research groups, post-operatory DH continues to be the most frequently reported adverse effect. In fact, a previous study investigating the correlation between bleaching efficacy and risk/intensity of post-operatory DH indicated, based on a multi-regression and logistic analysis, that the risk for the occurrence of DH was 120% more likely to precipitate from IPB than from at-home bleaching techniques. In addition, the intensity of painful symptoms were reported to be at least four times stronger for patients treated with IPB than those subjected to at-home treatments.

According to previous studies, the intensity (low, mild and severe) and duration (short-term or long-term) of DH precipitates directly from peroxide concentrations and exposure times used. Therefore, the behavior reported is expected because at-home bleaching gels are three-to-six times less concentrated than those used in IPB, were demonstrated to be less cytotoxic and to penetrate less into the tooth structure, thereby diminishing potential risks associated with the vitality of pulpal tissues. Despite these promising results, at-home techniques require long exposure times, and result in bleaching outcomes that are similar to those achieved with IPB. This critical scenario underscores the need for the development of techniques and products that are capable of resolving dental discolorations in a short period of time and without causing DH or negatively impacting the properties of teeth (surface, mechanical and biological).

The present disclosure therefore is directed to providing whitening agents that overcome the shortcomings of the currently available bleaching agents.

### BRIEF DESCRIPTION OF THE DRAWINGS

Several embodiments of the present disclosure are hereby illustrated in the appended drawings. It is to be noted however, that the appended drawings only illustrate several typical embodiments and are therefore not intended to be considered limiting of the scope of the inventive concepts disclosed herein.

FIG. 1 shows aspect images of experimental polymers without NF_TiO₂ (left) or with (right) 10% of NF_TiO₂. The incorporation of NF_TiO₂ in the concentration mentioned rendered experimental materials that were transparent, had pale-yellow color and were free of large agglomerates.

FIG. 2 shows specimens from G17-HP35+5NP+LT being subjected to IPB with visible light irradiation (LT). (HP=hydrogen peroxide, NP=nanoparticles).

FIG. 3 shows mean and standard error values of ΔE₀₀, ΔE_{ab} and ΔWI_{D} that were calculated considering the coordinate values collected before (T₀) and 14 days (T₄) after the last bleaching session with 6%, 15% and 35% HP without or with NF_TiO₂ (either 5 or 10%) incorporated.

FIG. 4 shows the temporal evolution (0, 10, 20 and 30 min) of pH values of the experimental gels (6%, 15% and 35% HP) with or without NF_TiO₂ and LT.

FIGS. 5A-H show ATR-FTIR spectra of specimens before (black curves, [To]) and 14 days after (dashed curves, [T₄]) being treated with experimental bleaching gels containing 6% HP and 0%, 5% or 10% of NF_TiO₂ and with or without LT. Controls (A-B), 6% HP w/o LT (C-E), 6% HP w/LT (F-H).

FIGS. 5I-T show ATR-FTIR spectra of specimens before (black curves, [To]) and 14 days after (dashed curves, [T₄]) being treated with experimental bleaching gels containing 15% HP or 35% HP and 0%, 5% or 10% of NF_TiO₂ and with or without visible light irradiation (LT). 15% HP w/o LT (I-K), 15% HP w/LT (L-N), 35% HP w/o LT (O-Q), 35% HP w/LT (R-T).

FIG. 6A shows mean and standard error values acquired from the area under the curves corresponding to CO₃² υ2 (A) and PO₄³ υ1 (B) and υ2 (C) or the mineral ratio (D) of the integrated areas of CO₃² υ2 to PO₄³ υ1, υ2 contours. These values were calculated before and 14 days after bleaching, taking into consideration all the bleaching protocols adopted in the present work.

FIG. 6B shows mean and standard error values acquired from the area under the curves corresponding to PO₄³ υ2 (C) or the mineral ratio (D) of the integrated areas of CO₃² υ2 to PO₄³ υ1, υ2 contours. These values were calculated before and 14 days after bleaching, taking into consideration all the bleaching protocols adopted in the present work.

FIGS. 7A-P show images acquired using atomic force microscopy showing illustrative areas of the enamel before (TO) and after 14 days (T₄) from bleaching with 6% HP and 0%, 5% or 10% of NF_TiO₂ in the absence or presence of LT. Controls (A-D), 6% HP w/o LT (E-F, I-J, M-N), 6% HP w/LT (G-H, K-L, O-P).

FIGS. 7Q-PP show images acquired using atomic force microscopy showing illustrative areas of the enamel before (TO) and after 14 days (T₄) from bleaching with 15% or 35% HP and 0%, 5% or 10% of NF_TiO₂ in the absence or presence of LT. 15% HP w/o LT (Q-R, U-V, AA-BB), 15% HP w/LT (S-T, X-Z, CC-DD). 35% HP w/o LT (EE-FF, II-JJ, MM-NN), 35% HP w/LT (GG-HH, KK-LL, OO-PP).

FIG. 8 shows temporal evolution (0 min, 2 min, 4 min and 6 min) of RLU signals after the addition of D-Luciferin substrate to 24-hour Streptococcus mutans (JM10) biofilms.

FIGS. 9A-R show 3-D reconstructions of the biofilms with concurrent staining observed under laser confocal microscopy only 14 days after bleaching with 6%, 15% and 35% HP with or without incorporation of 5% and 10% NF_TiO₂. Controls (A-B), 6% HP w/o LT (C-E), 6% HP w/LT (F-H), 15% HP w/o LT (I-J), 15% HP w/LT (K-L), 35% HP w/o LT (M-O), 35% HP w/LT (P-R).

### DETAILED DESCRIPTION

Disclosed herein is a highly efficient dental bleaching (whitening) agent having biomimetic properties. In one embodiment the dental bleaching agent may be applied to teeth in in a dental office (“in-office” or “chair-side”). This product represents a vertical advancement in the clinical practice of dentistry because patients treated with the disclosed bleaching agents result in excellent bleaching outcomes in teeth while displaying unaltered surface and chemical properties and reduced post-operatory dentin hypersensitivity. The disclosed bleaching agent allows for esthetic outcomes that are comparable to those attained with commercially-available bleaching gels containing 35% of hydrogen peroxide (HP) but at significantly lower concentrations. Because of the low HP concentration used, surfaces and chemical make-up are maintained, which is important to maintain the health of treated tissues. The novel bleaching agents disclosed herein are also able to increase the mineral content of treated teeth, thereby making them more resistant to organic acids. No product currently available in the market is capable of achieving similar results. Patients treated with the present technology will display teeth that are very white and more resistant to bacterial attacks, without the negative effects of prior teeth bleaching methods. In certain non-limiting embodiments, the present disclosure includes dental bleaching agents containing synthetic hydrophilic polymers (e.g., Carbomer 940) or natural hydrophilic polymers (e.g., Hyaluronic acid), hydrogen peroxide (e.g., 6 wt %), and doped titanium dioxide (TiO₂) nanoparticles (e.g., doped with nitrogen and fluorine).

Utilization of co-doped metal oxide nanoparticles, synthesized using solvothermal reactions, for dental bleaching applications is at least one novel aspect of the present disclosure. Result provided herein show, for example, that bleaching gels containing 6% HP and 5% co-doped TiO₂ nanoparticles were capable not only achieving esthetic outcomes that were comparable to those attained with bleaching gels containing 35% HP (which is the “gold standard” of previously available dental bleaching agents), but were also demonstrated to not adversely impact the chemical make-up of treated enamel, and were shown to mineralize the tooth structure, both of which are properties that have never been reported in dentistry as co-occurring in dental bleaching agents. The utilization of the hyaluronic acid as polymeric matrix is also novel and has never been reported in Dentistry. In combination, the results reported indicate that novel bleaching agents disclosed herein can resolve mild-to-severe dental discolorations in a short period of time, can result in lower levels of post-operatory dentin hypersensitivity, and do not alter any surface or chemical properties of treated tissues.

Before further describing various embodiments of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning, and the embodiments and examples are meant to be exemplary, not exhaustive. Also, it is to be understood that the phraseology and terminology employed herein is for the purpose of description only and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. Thus, while the compounds, compositions, and methods of the present disclosure have been described in terms of particular embodiments, it will be apparent to those of skill in the art that variations may be applied to the compounds, compositions, and methods and in the steps or in the sequence of steps of the methods described herein without departing from the concept, spirit, and scope of the inventive concepts.

All patents, published patent applications, and non-patent publications including published articles mentioned in the specification or referenced in any portion of this application are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. Where used herein, the specific term “single” is limited to only “one.”

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

As used in this specification and claims, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB.

Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The terms “about” or “approximately,” where used herein when referring to a measurable value such as an amount, a temporal duration, and the like, is meant to encompass, for example, variations of +20% or +10%, or +5%, or #1%, or +0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment. Further, all references to one or more embodiments or examples are to be construed as non-limiting to the claims.

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds or conjugates of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

The term “nanoparticle,” as used herein, generally refers to a particle having a diameter, such as an average diameter, from about 1 nm, to about 5 nm, to about 10 nm, to about 50 nm up to about 1000 nm, including, for example, particles having an average diameter of 5 nm, to 10 nm, up to about 100 nm, up to about 200 nm, up to about 300 nm, up to about 400 nm, up to about 500 nm, up to about 600 nm, up to about 700 nm, up to about 800 nm, or up to about 900 nm or more. The particles can have any shape. Nanoparticles having a spherical shape may be referred to as “nanospheres”.

The term “microparticle,” as used herein, generally refers to a particle having a diameter, such as an average diameter, from about 1 micron (micrometer) to about 100 microns, for example including particles having an average diameter from about 1 micron to about 50 microns, from about 1 micron to about 40 microns, from about 1 micron to about 30 microns, from about 1 micron to about 25 microns, from about 1 micron to about 20 microns, from about 1 micron to about 10 microns, or from about 1 to about 5 microns. The microparticles can have any shape. Microparticles having a spherical shape may be referred to as “microspheres”.

The term “active agent” as used herein is intended to refer to a substance which possesses a biological activity relevant to the present disclosure, and particularly refers to therapeutic and diagnostic substances which may be used in methods described in the present disclosure. “Biologically active” refers to the ability of a substance to modify the physiological system of a cell, tissue, or organism without reference to how the substance has its physiological effects.

The term “effective amount” refers to an amount of an active agent which is sufficient to exhibit a detectable therapeutic or treatment effect in a subject without excessive adverse side effects (such as substantial toxicity, irritation, and allergic response) commensurate with a reasonable benefit/risk ratio when used in the manner of the present disclosure. The effective amount for a subject will depend upon the subject's type, size, and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, “pure,” or “substantially pure” means an object species (e.g., an imaging agent) is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., an imaging agent) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

“Treatment” refers to therapeutic treatments. “Prevention” refers to prophylactic or preventative treatment measures. The term “treating” refers to administering the composition to a patient for therapeutic purposes.

The term “topical” is used herein to define a mode of administration on or through a surface, such as but not limited to, a material that is administered by being applied externally to a tooth or gum epithelial surface.

The term “mineralization” where used herein refers to the addition of minerals (e.g., calcium and/or fluorine) to the enamel, dentin or cementum of the teeth, while “demineralization” refers to a loss of (decrease in) minerals from enamel, dentin, or cementum. One measure of mineralization is an increase in the phosphate: carbonate ratio of the teeth after completion of a treatment with the presently disclosed dental bleaching composition. A non-limiting example of how the phosphate: carbonate ratio in a tooth can be quantitavely determined is by using 3-D Cone Beam Computed Tomography and the mineral density of hard tissues treated (enamel and dentin).

The term “hypersensitivity”, where used herein refers to a condition in which a tooth is highly sensitive to, e.g., coldness, heat, salt, acid, or touch. A standard method for measuring sensitivity in a tooth is by gentle touching or tapping with a probe, or blowing air or water over the tooth and rating the response of the patient to the stimulus on a scale of 0-3, wherein a rating of 2 or 3 is considered to be hypersensitive.

The term “in need of dental repair” where used herein refers to one or more teeth which require some type of filling, bonding, or adhesion. Examples include a tooth with a cavity or a chipped tooth, attaching a crown or veneer, or filling a crack.

In at least certain embodiments, the presently disclosed dental compositions do not comprise a dental adhesive or bonding agent such as Optibond®.

The terms “therapeutic composition,” and “pharmaceutical composition” refer to an active agent-containing composition (e.g., a composition comprising doped TiO₂ nanoparticles, such as NF_TiO₂ NPs or Nag_TiO₂ NPs) that may be administered to or used in a subject by any method known in the art or otherwise contemplated herein, wherein administration or use of the composition brings about an effect or result as described elsewhere herein. In addition, the compositions of the present disclosure may be designed to provide delayed, controlled, extended, and/or sustained effects using formulation techniques which are well known in the art.

Turning to the description of several particular embodiments, various bleaching formulations were made for experimentation by combining NF_TiO₂ nanoparticles (5% w/v and 10% w/v) and varying concentrations of HP (6% w/v, 15% w/v or 35% w/v) with polymer gel. The bleaching efficacy of the resulting gels on bovine enamel chemical make-up and surface roughness was tested. Additional analyses were focused on revealing how experimental bleaching protocols affected the metabolism and the components of single-species biofilms using a minimally invasive, real-time and high throughput bioluminescence assay and a concurrent staining technique along with confocal microscopy, respectively. In non-limiting embodiments, the bleaching composition may comprise a weight to volume ratio (w/v) of peroxide (e.g., HP or CP) to polymeric matrix (carrier) material in a range of 0.1% to 35%, 0.1% to 30%, 0.1% to 25%, 0.1% to 30%, 1% to 35%, 1% to 30%, 2% to 25%, 3% to 20%, 3% to 18%, 4% to 18%, 4% to 15%, 5% to 35%, 5% to 25%, 5% to 15%, 5% to 12%, 6% to 35%, 6% to 30%, or 6% to 25%, 6% to 20%, 6% to 15%, 6% to 12%, 6% to 10%, or 6% to 8%, for example. In non-limiting embodiments, the bleaching composition may comprise a weight to volume ratio (w/v) of doped TiO₂ to polymeric matrix material in a range of 1% to 50%, 1% to 30%, 2% to 25%, 3% to 20%, 3% to 18%, 3% to 15%, 3% to 12%, 3% to 10%, 3% to 8%, 4% to 18%, 4% to 15%, 4% to 12%, 4% to 10%, 4% to 8%, 5% to 18%, 5% to 15%, 5% to 12%, 5% to 10%, 5% to 8%, 6% to 15%, 6% to 12%, or 6% to 10%, for example.

Polymer and non-polymer thickening-agents that may be used in the dental bleaching compositions of the present disclosure include but are not limited to those shown in U.S. Pat. Nos. 4,405,599; 4,528,180; 4,687,663; 4,839,157; 4,849,213; 5,098,303; 5,234,342; 5,290,566; 5,376,006; 5,631,000; 5,718,886; 5,725,843; 5,785,527; 5,851,512; 5,858,332; 5,985,249; 6,231,343; 6,306,370; 6,309,625; 6,312,671; 6,322,774; 6,368,576; 6,387,353; 6,458,340; 6,485,709; 6,500,408; 6,503,485; 6,558,654; 6,669,930; 6,908,607; 7,011,523; 7,060,256; 8,574,555; 8,728,447; 8,926,778 and 8,980,231. Each of these patents is expressly incorporated herein by reference in its entirety.

In general, the dental bleaching composition may comprise a tackifying agent, as well as the peroxide bleaching agent, the gel-forming thickening agent, and the nitrogen-fluorine co-doped titanium dioxide nanoparticles. The bleaching composition may optionally include an adhesive agent. A non-limiting example of a tackifying agent that can be used in the present compositions is polyvinyl pyrrolidone (PVP). Non-limiting examples of polyvinyl pyrrolidone polymers that have been used in formulating dental bleaching gels include Kollidon 30, a polyvinyl pyrrolidone polymer sold by BASF having a molecular weight of 50,000, Kollidon VA 60, a polyvinyl pyrrolidone polymer having a molecular weight of 60,000, and Kollidon 90 F, a polyvinyl pyrrolidone polymer having a molecular weight of 1.3 million. Other tackifying agents that may be used herein include, but are not limited to, carboxypolymethylene (e.g., CARBOPOL, sold by Novean, Inc.), polyethylene oxide (e.g., POLYOX, made by Union Carbide), polyacrylic acid polymers or copolymers (e.g., PEMULEN, sold by Novean, Inc.), polyacrylates, polyacrylamides, copolymers of polyacrylic acid and polyacrylamide, PVP-vinyl acetate copolymers, carboxymethylcellulose, carboxypropylcellulose, polysaccharide gums, proteins, and the like. The one or more tackifying agents may be included in an amount in a range of about 1% to about 50% by weight of the dental bleaching gel, or in a range of about 3% to about 30% by weight of the dental bleaching gel, or in a range of about 5% to about 20% by weight of the dental bleaching composition.

The dental bleaching composition will typically include one or more liquid or gel carriers or vehicles into which the other components are dispersed. Examples of liquid or gel carriers or vehicles include, but are not limited to, water, alcohols (e.g., ethyl alcohol), and polyols (e.g., glycerin, sorbitol, polyethylene glycol, polyethylene oxide, propylene glycol, and polypropylene glycol). The carrier or vehicle will typically comprise the balance of components in the dental bleaching composition. The dental bleaching composition may optionally include other components as desired to yield a bleaching gel having desired properties. Examples include stabilizing agents (e.g., EDTA), neutralizing agents (e.g., sodium hydroxide), thickening agents (e.g., fumed silica), desensitizing agents (e.g., potassium nitrate, other potassium salts, citric acid, citrates, and sodium fluoride), remineralizing agents (e.g., sodium fluoride, stannous fluoride, sodium monofluorophosphate, and other fluoride salts), antimicrobial agents (e.g., chlorhexidine, troclosan, and tetracycline), antiplaque agents, anti-tartar agents (e.g., pyrophosphates salts), other medicaments, flavorants, sweeteners, and the like.

An exemplary thickening agent is Pluronic, a copolymer of polyethylene oxide and polypropylene oxide, for example, a hydrophilic variety of Pluronic is Pluronic F127 (also known as Poloxamer 407) a trade name of BASF, which gels at room temperature (at about 20° C.) in water. This gelling agent is very stable and not prone to oxidation by H₂O₂. Pluronic also acts as a surfactant, which helps to remove extrinsic stains from teeth. In addition to Pluronic, a small amount of glycerol and sodium chloride (at 5% concentration) may be used to impart optimal viscosity to the formulation. It is to be noted that more gelling agent (Pluronic F 127) would be required to gel a hydrogen peroxide solution than would be required to gel a pure water-based solution, due to the gel-weakening effects imparted by the additives in the hydrogen peroxide solution. The dispersed phase of the gel may be an organic polyol. Examples of acceptable organic polyols are propylene glycol and glycerin. An example of an polyol component is glycerin (C₃H₈O₃), a commercially available trihydric alcohol that is also known by the names glycerol, glycyl alcohol, 1,2,3-propanetriol, and trihydroxypropane. The organic polyol may be employed in an amount of at least 25% by weight of the gel. The exact amount of organic polyol employed in the gel may vary in almost direct relation to the amount of bleaching agent employed in the gel. In a non-limiting example, the dental bleaching composition may include up to approximately 60% by weight organic polyol.

Thickeners that may be used include the crosslinked polyacrylic resins sold by B.F. Goodrich under the tradenames Carbopol® 1342, Carbopol® 1382, Carbopol®, ETD® 2020, Carbopol®, and Ultrez® 10. These polymers are either homopolymers of acrylic acid crosslinked with allyl sucrose, polyalkyl ethers of divinyl glycol, or allyl pentaerythritol or similarly crosslinked copolymers of acrylic acid with minor levels of long chain alkyl acrylate comonomers. These polymers swell in water up to 1000 times their original volume (and ten times their original diameter) to form a gel when exposed to a pH environment above 4.0-6.0. Carbopol® thickeners are highly resistant to hydrolysis and oxidation under normal conditions.

The bleaching composition may include a stabilizing agent utilized in the aqueous gel, for example in an amount ranging from 0.05% to 1% by weight of the aqueous gel. The stabilizing agent may be, for example, an aminocarboxylic acid or salt thereof, or an alkali and/or alkali earth metal salt thereof. Suitable aminocarboxylic acids include trans-1,2-cyclohexylene dinitrilotetraacetic acid (CDTA), ethylenediamine tetraacetic acid (EDTA), N-(2-hydroxyethyl)ethylenediamine triacetic acid (HEDTA), Nitrilotriacetic acid (NTA), diethylene triamine pentaacetic acid (DTPA), triethylene tetraamine hexaacetic acid (TTHA), ethylene glycol bis (2-aminoethylether)tetraacetic acid (GEDTA), CaNa₂EDTA, Na₂EDTA, Na₄EDTA, HEDTA, and Na₃HEDTA.

### Examples

Certain embodiments of the present disclosure will now be further discussed in terms of several specific, non-limiting, examples. The examples described below will serve to illustrate the general practice of the present disclosure, it being understood that the particulars shown are merely exemplary for purposes of illustrative discussion of particular embodiments of the present disclosure only and are not intended to be limiting of the claims of the present disclosure.

### Materials and Methods

### Specimen Preparation and Experimental Groups

Bovine central incisors (n=300) were extracted and stored in thymol solution (0.1%, 8° C.) for no longer than two months. Following, remnants of periodontal ligament and attached organic tissues were removed using scalpels. After that, roots were horizontally sectioned 2.0 mm apically from the enamel-cementum junction using a high-speed diamond saw (KG Sorensen, Cotia, SP, Brazil) under copious water irrigation. Squared shaped specimens (Enamel/dentin; Area=36.0 mm², thickness=3.0 mm) were then obtained from the central area of bovine crowns using a metallographic diamond saw (Isomet, Buehler; Lake Bluff, IL, USA) under abundant irrigation with water.

Following, blocks obtained were flattened and finished using a rotary polisher (Arotec, São Paulo, SP, Brazil) and abrasive disks (600- and 1,200-Grit, Norton Saint-Gobain, Guarulhos, SP, Brazil) before being polished using diamond suspensions (1 μm, 0.50 μm and 0.25 μm, Erios, São Paulo, SP, Brazil) and polishing cloths (3M Brazil, Sumaré, SP, Brazil). Prepared specimens were then subjected to Knoop microhardness testing (diamond indenter, 50.0 g load, 5 s/indentation, 3 indentations/sample, 100 μm apart; Future Tech FM-ARS, Tokyo, Japan). Specimens (n=200; 10/group) with standardized microhardness (296.07±29.60) were then randomly distributed into twenty experimental groups (“G”) based on content (w/v) of hydrogen peroxide (HP6=6% HP, HP15=15% HP, and HP35=35% HP), and nanoparticles (NP5=5% NP, NP10=10% NPs) and whether visible light irradiation was applied (LT), as follows: G1-No HP, NPs or LT, G2-LT only, G3-HP6, G4-HP15, G5-HP35, G6-HP6+LT, G7-HP15+LT, G8-HP35+LT, G9-HP6+5NP, G10-HP15+5NP, G11-HP35+5NP, G12-HP6+10NP, G13-HP15+10NP, G14-HP35+10NP, G15-HP6+5NP+LT, G16-HP15+5NP+LT, G17-HP35+5NP+LT, G18-HP6+10NP+LT, G19-HP15+10NP+LT, and G20-HP35+10NP+LT.

### Nanoparticle Synthesis

A detailed description of the synthesis of NF_TiO₂ nanoparticles and other single-doped and co-doped TiO₂ nanoparticles is provided in US Published Patent Application 2020/0085698, the entirety of which is hereby expressly incorporated by reference herein.

In a particular non-limiting example of TiO₂ nanoparticle synthesis, a solution comprised of 1.7 g of Ti(IV)-butoxide (Aldrich, 97%), 4.6 g ethanol (Decon Labs, 100%), 6.8 g oleylamine (Aldrich, 70%), and 7.1 g oleic acid (Aldrich, 90%) was prepared, then mixed with 20 mL of 4% H₂O in ethanol (18-MΩ Milli-Q; Decon Labs). Each solution was clear before mixing, but the final mixture immediately clouded due to formation of micelles and likely some hydrolysis. This solution was then split into two portions (around 20 mL/portion), and each portion was placed into a high-pressure reaction vessel (Paar Series 5000 Multiple Reactor System) and reacted at 180° C. for 24-hours. The vessels were stirred via external magnetic field and Teflon-coated stir bars. The reaction vessels were Teflon-lined. Upon cooling, the solutions were decanted and rinsed 3 times with anhydrous ethanol to remove extraneous surfactants resulting in pure TiO₂ NPs which were readily dispersible into 20-30 mL ethanol, but did not form clear solutions. The TiO₂ NPs formed were stored in ethanol.

NF_TiO₂ NPs can be formed following the above reaction steps using Ti(IV)-butoxide. F and N are provided by adding ammonium fluoride as the dopant sources in a wt:wt:wt N:F:Ti ratio of, for example, 1:1:18, which provides a 5%/5%/90% N/F/Ti composition. For example in one embodiment, components sufficient to provide 0.085 g N and 0.085 g F can be combined with a component comprising Ti (e.g., Ti(IV)-butoxide) are used. The TiO₂ NPs form as in the process above but with the N and F dopants in place.

In another embodiment of a process for forming N and F-doped NPs, a solution of 1.7 g of Ti(IV)-butoxide (Ti(OBu)₄) (Aldrich, 97%), 4.6 g C₂H₅OH (Decon Labs, 200 proof), 6.8 g C₁₈H₃₅NH₂ (Aldrich, 70%), 7.1 g C₁₈H₃₄O₂ (Aldrich, 90%) and 5% of NH₄F (based on Ti content; crystalline, ACS, Alfa Aesar) was prepared and then mixed with an ethanol-water solution (4%, 18-Milli-Q; total weight=13.10 g). Solutions prepared were transparent before mixing, however, the final solution clouded instantaneously after mixing due to hydrolysis and some micelle formation. The final solution was placed into a high-pressure reaction vessel (Borosilicate Glass-lined; Paar Series 4593, Bench Top Reactor System), reacted (180° C., 24 hours, 15 psi) and stirred via external shaft coupled to a turbine impeller (280 rpm). At the end of the 24-hour cycle, the solution was removed from the reaction vessel, and transferred to a 50 mL falcon tube with a certain amount of ethanol (200-proof, Decon Labs). The solution was centrifuged during 15 min at 8,000 rpm. Such procedure was repeated for two additional times, using 20 mL of ethanol.

### Polymer Synthesis and Incorporation of NF_TiO₂

Experimental bleaching gels were formulated by mixing a commercially-available hydrophilic polymer (12.5 g, Carbomer 940 NF, Spectrum, Gardena, CA) to an aqueous solution (distilled, 400 mL, pH=11) containing KOH (60%, 20 mL) using a planetary and orbital stand-alone mixer (1 cycle at 2,000 rpm for 2 minutes, 2 additional cycles at 2,500 rpm, 3 minutes each; Speed Mixer, DAC 400.1 FVZ, FlackTek Inc, Laudrum, SC, USA). Immediately after mixing, the resulting polymer (pH ˜6) was observed to be transparent and free of any undissolved polymer (white agglomerates). The experimental polymer was then stored in a black container for at least 24 hours (refrigerator, 8° C.).

Two aliquots (1 mL and 2 mL, respectively) of nitrogen and fluorine co-doped titanium dioxide nanoparticles (NF_TiO₂, ˜40 mg/mL) suspended in ethanol were placed in individual plastic tubes and were centrifuged (8,000 rpm, 5 min) in preparation for polymer incorporation procedures. Ethanol-free NF_TiO₂ were then individually mixed into 20 g of the experimental polymer to render gels containing either 5% or 10% of NF_TiO₂. Each nanofilled gel was then mixed at 2,450 rpm for 20 s (Speed Mixer, DAC 400.1 FVZ, FlackTek Inc, Laudrum, SC, USA). The final gel continued to be transparent and free of visible agglomerates, but its color became pale-yellow due to the successful incorporation and dispersion of NF_TiO₂.

Incorporation of Hydrogen Peroxide (H₂O₂)

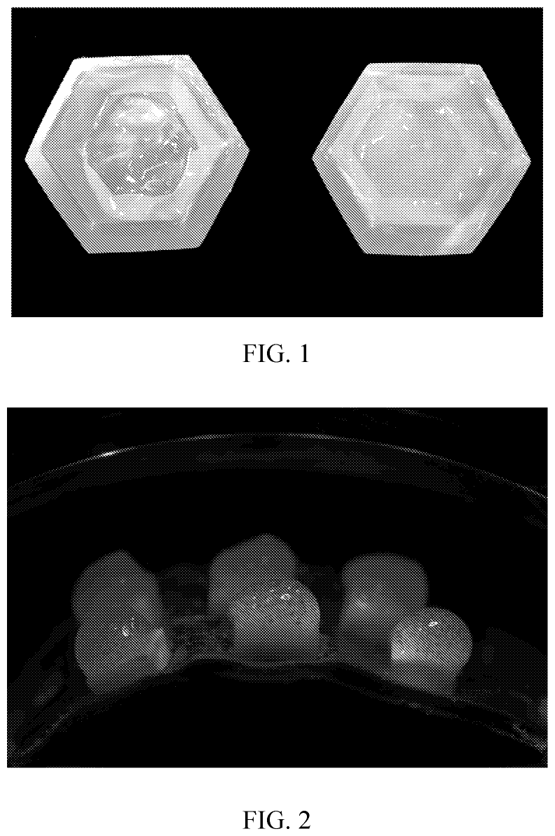

Immediately before utilization, experimental gels (either 1 g or 1.5 g, depending on H₂O₂:polymer ratio) with or without NF_TiO₂ (either 5% or 10%) were manually mixed (1:2 [6% or 15% H₂O₂] or 2:3 [35% H₂O₂]) with 1 mL of hydrogen peroxide following previous published protocols (FIG. 1). The rationale for the utilization of two distinct H₂O₂: polymer ratios was based on the need to achieve comparable viscosities for all experimental materials investigated.

### Bleaching Protocols

Experimental bleaching protocols investigated consisted of three sessions (T₁, T₂ and T₃, 7-days apart). Each 30-minute session was based on a single application of the proper experimental gel (with or without nanoparticles) combined or not with continuous visible light irradiation (20 cycles of 1 min, 30 second interval between irradiation cycles; 405±15 nm, 1.2 W/cm², emission window area=10.7 cm²) in function of experimental groups (G1 to G20, see above). FIG. 2 illustrates specimens being subjected to dental bleaching procedures modulated by experimental bleaching gels and visible light irradiation (G17-HP35+5NP+LT). After each session, specimens from all groups were stored (37° C., dark conditions) in artificial saliva (1.5 mM calcium chloride [CaCl₂)], 0.9 mM sodium phosphate [NaH₂PO₄], 0.15 mM potassium chloride [KCl, pH 7.0]). After the third session (T₃), specimens were then stored in artificial saliva for 14 days.

### Objective Colorimetric Evaluation

The objective colorimetric evaluation (in terms of L*, a*, and b*) was performed before the first bleaching session (baseline, To) and 14 days after the last bleaching session (T₄) using a hand-held digital spectrophotometer (Vita EasyShade, VITA Zahnfabrik H. Rauter GmbH & Co. KG, Bad Sackingen, Germany). Variation of color (T₄−T₀) was determined according to the CIELab system (Comission Internationale de l'Eclairage, L*, a*, b*) and using the formulae for ΔE_{ab} (eq. 1),²⁹ ΔE₀₀³⁰ (eq. 2) and ΔWI_{D} (eq. 3),³¹ as follows:

$$Δ {E}_{ab} = \sqrt{{( Δ L )}^{2} + {( Δ a )}^{2} + {( Δ b )}^{2}} \qquad ( eq . 1 )$$

$$Δ {E}_{00} = \sqrt{{( \frac{Δ {L}^{′}}{{K}_{L} {S}_{L}} )}^{2} + {( \frac{Δ {C}^{′}}{{K}_{C} {S}_{C}} )}^{2} + {( \frac{Δ {H}^{′}}{{K}_{H} {S}_{H}} )}^{2} + RT \cdot ( \frac{Δ {C}^{′}}{{K}_{C} {S}_{C}} ) \cdot ( \frac{Δ {H}^{′}}{{K}_{H} {S}_{H}} )} \qquad ( eq . 2 )$$

$${WI}_{D} = 0.55 {L}^{*} - 2.32 {a}^{*} - 1.1 {b}^{*} \qquad ( eq . 3 )$$

### pH Analysis

The temporal evolution (10-minute increments, total time=30 min) of pH was determined for experimental bleaching gels (1 g of each, with or without nanoparticles) irradiated or not with visible light using a calibrated pH meter (AB150, Accumet, Fisher-Scientific, Hampton, NH, United States) to determine the impact of pH on properties investigated.

### Mineral Content Evaluation

Infrared spectra of bovine enamel at T₀ and T₄ were acquired at three locations per specimen using an infrared spectrometer (IS50, Nicolet Madison, WI, USA; scanning parameters: 500-4,500 cm⁻¹; resolution 4 cm⁻¹, 10 internal scans per spectrum/per location) coupled to a heated attenuated total reflectance (ATR) monolithic diamond crystal (Golden Gate, Specac). A method previously described was utilized to guarantee that ATR-FTIR measurements were performed exactly at the same locations in each specimen. Enamel spectra (at T₀ and T₄) from each specimen were corrected for the presence of water before being subjected to baseline correction and normalization procedures using the OMNIC software (v7.0 Madison, WI, USA). The areas under the peaks corresponding to CO₃² υ2 (886 cm⁻¹), PO₄³ υ1 (996 cm⁻¹) and PO₄³ υ2 (1,410-1,460 cm⁻¹) were calculated before and after experimental treatments. The mineral composition of enamel (in terms of carbonate: phosphate mineral ratio) was determined by integrating the areas under the curves of CO₃² υ2 and PO₄³ (υ1 and υ2).

### Topography Assessment

An atomic force microscope (Nanoscope V, Bruker, Billerica, MA, United States) in ScanAssist mode coupled with silicon nitride probes (aluminum-coated, triangular, radius=2 nm, spring constant=0.4 N/m, Bruker) was used to reveal topographical aspects of specimens (n=1/group) at T₀ and T₄. Images (A=25 μm²; 512×512 lines) were acquired (at same locations at T₀ and T₄) using a scan rate of 0.8 Hz. Images were then flattened before topographical parameters of interest (Rₐ [roughness average] and R_{q} [root mean square roughness]) were determined using the Nanoscope software (v9.0, Bruker)

### Metabolic Status of Non-Disrupted Biofilms

A minimally invasive, real-time and high throughput bioluminescence assay was used to determine the metabolic status of non-disrupted Streptococcus mutans biofilms grown of the surfaces of specimens treated according to experimental groups described above (Specimen Preparation and experimental groups). In brief, planktonic cultures of Streptococcus mutans (JM10) were grown overnight (16 hours) in a liquid culture medium (THY) at oral temperature. Cultures having optical density higher than 0.900 (at 600 nm; corresponding to 6.43 e⁺¹² CFU/mL) were used as inoculum to grow biofilms. S. mutans biofilms were then grown (24 hours, anaerobic conditions, 37° C.) on the surfaces of sterile specimens (UV-sterilized, 254 nm, 800,000 μJ/cm², UVP Crosslinker, model CL-1000, UVP, USA) using inoculated biofilm growth media (0.65×THY, 1:50 dilution, 1.0 mL/well) supplemented with sucrose (1%, w/v). After 24 hours, biofilms were replenished with 1.0 mL of fresh 1×THY+1% (w/v) glucose recharge medium and were incubated (37° C., 1 hour) before being transferred into the wells of sterile white 24-well plates containing 1.0 mL of fresh 0.65×THY+1% (w/v) sucrose medium. An aqueous solution (100 mM) of D-Luciferin suspended in citrate buffer (0.1 M, pH 6.0) was added by a Synergy-HT system (Biotek, USA) to the wells containing both the specimens and biofilms in recharge medium (2:1 ratio [v/v/] inoculum to D-Luciferin). The metabolic activity of non-disrupted biofilms was assessed (in terms of RLUs) at 590 nm in 2-min increments (total of 6 minutes) after the addition of D-Luciferin.

### Staining and Confocal Microscopy

A concurrent staining method was used to illustrate the impact of experimental bleaching treatments on the distribution of biofilm components such as nucleic acid, proteins and extracellular polymeric substances (EPS). To achieve this goal, an additional set of specimens (n=1/group) were prepared and bleached according to methods described above. Biofilms were grown on the surfaces of sterile specimens using the methods described above. After the 24-hour growth period, biofilms were washed with PBS (3×, pH 7.4, 25° C., 15 s/wash) to remove non-adherent cells. Following, biofilms were concurrently stained with Alexa Fluor® 647 conjugate of Concanavalin A (Invitrogen, USA; 250 μg/mL), Syto 9 (Molecular Probes, USA; 10 μM), and Sypro Red (Invitrogen, USA; 10×). Biofilms were kept hydrated in sterile ultra-pure water and protected from light until confocal microscopy. Images of biofilms were acquired using a TCS-SP2 MP confocal laser scanning microscope (CLSM, Leica Microsystems, Inc., USA) with Ar (488 nm) and He/Ne (543 and 633 nm) lasers for the excitation of the fluorescent stains at three different locations of the specimens' enamel surface. A 63× water immersion microscope objective lens was used. Serial optical sections were recorded from the surface of specimens to the top of biofilms at 0.6 μm intervals in the Z-direction. Representative 3-D images of the biofilms were generated using Volocity software (PerkinElmer, USA) to allow the visualization of the distribution of the nucleic acids, proteins, and EPS components of biofilms.

### Results

### Bleaching Efficacy

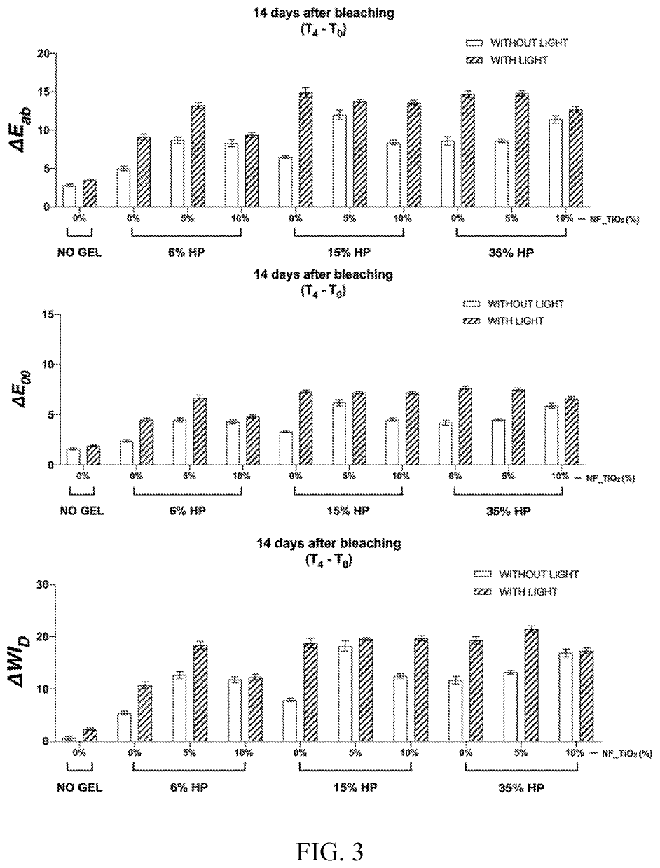

The results of the objective color analysis are shown in FIG. 3 (A-F) in terms of ΔE₀₀, ΔE_{ab} and ΔWI_{D} at T₄. The findings shown in FIG. 3 demonstrate that experimental bleaching protocols modulated by gels containing 6%, 15% and 35% of H₂O₂ (without NP or LT) displayed mean values of ΔE₀₀, ΔE_{ab} and ΔWI_{D} that were higher when compared to those of the control group (no treatment, with or without LT). These results also indicate that ΔE₀₀, ΔE_{ab} and ΔWI_{D} values vary with HP concentrations, and bleaching outcomes, can be rank ordered in terms of increasing efficacies where HP6<HP15<HP35, respectively. Even though a similar trend is observed when bleaching protocols are modulated by experimental bleaching gels containing 6%, 15% and 35% of HP (without NP) and visible light irradiation (405 nm±15 nm), ΔE₀₀, ΔE_{ab} and ΔWI_{D} values are higher than those from bleaching protocols with no light irradiation, thereby indicating that visible light irradiation can be used to increase the efficacy of dental bleaching procedures. The combination HP and NP is shown to further increase the efficacy of experimental bleaching protocols (with or without LT), as denoted by ΔE₀₀, ΔE_{ab} and ΔWI_{D} mean values that are higher than those previously discussed. These findings indicate that the incorporation of NP (in 5% and 10%, v/w) improved not only the dissociation of HP into ROS but could also be contributing factor in the generation of longer-lived species of oxygen.

### Analysis of pH

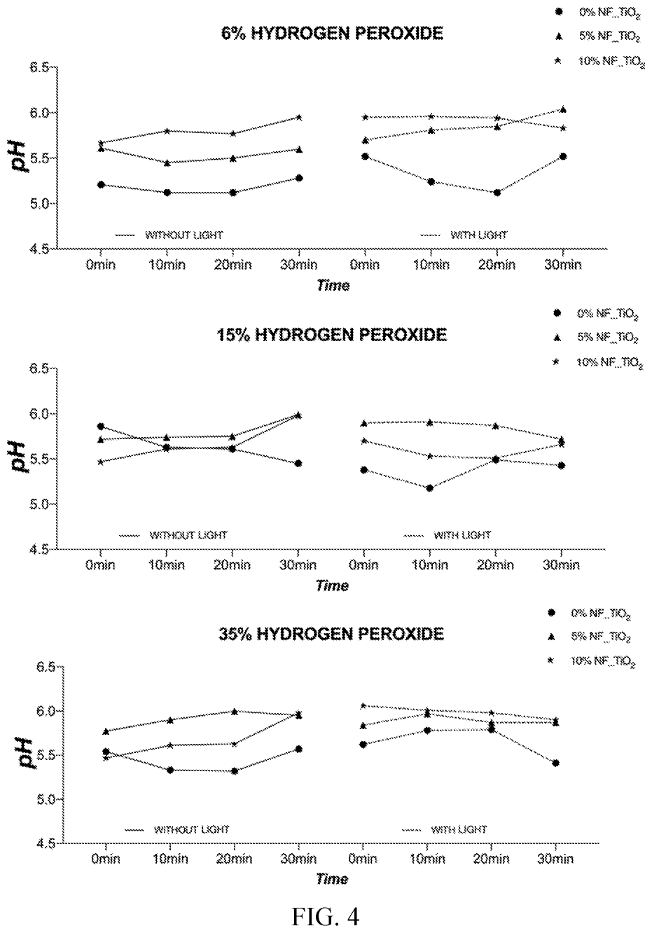

The graphs below (FIG. 4A-C) illustrate the temporal evolution of pH for experimental gels (6%, 15% and 35% HP) with or without NF_TiO₂ (5% and 10%). It is possible to observe that the experimental gels (6%, 15% and 35% HP) without the incorporation of NF_TiO₂ displayed pH values (=5.0, 6% HP+LT at 20 min) that were lower when compared to those containing NF_TiO₂. Such behavior was observed to be consistent throughout the observation time (at 0, 10, 20 and 30 min), and to depend mostly on NF_TiO₂ concentrations, thereby indicating that the incorporation of NF_TiO₂ acts to reduce the risk for enamel demineralization. The results reported have also indicated that such behavior is not influenced by visible light irradiation, and that nanofilled bleaching gels displayed comparable pH values after 30 minutes.

### Mineral Content Evaluation

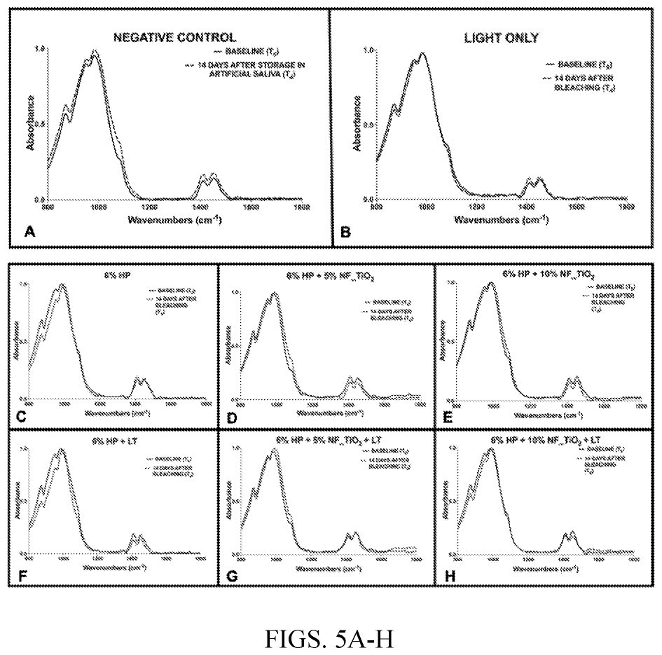

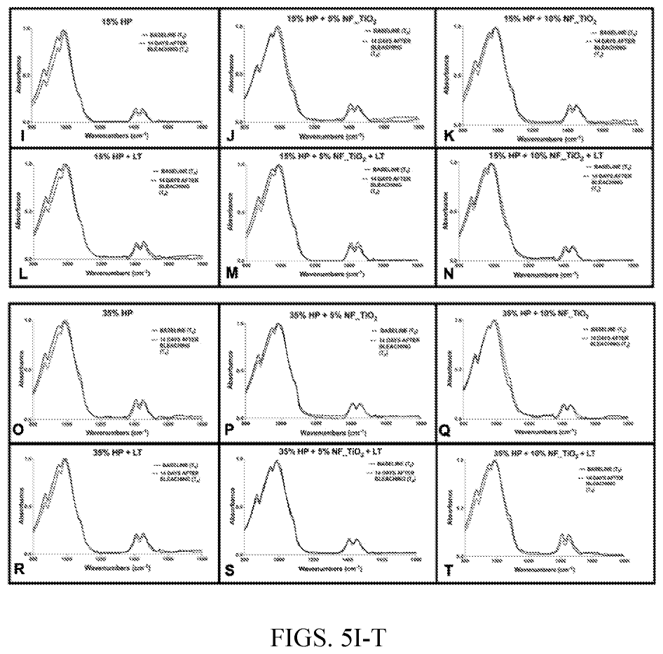

FIGS. 5A-5B (A-T) illustrate the results from the ATR-FTIR analysis of the mineral content of enamel before (baseline [T₀], black curves) and after bleaching (14 days after [T₄], red curves). FIG. 5A (A) indicates (in terms of normalized absorbance values) that specimens pertaining to G1 (negative control) displayed spectra characterized by absorbance values that were slightly higher at T₄ (wavenumbers from 800 cm⁻¹ to 1,150 cm⁻¹ and from 1,350 cm⁻¹ to 1,550 cm⁻¹) and by a larger spectral bandwidth at absorbance values between 0.0 and 0.5. In combination, these results indicate that the mineral content of enamel were not altered by storing specimens in artificial saliva for the duration of the experiment. A similar behavior was observed in FIG. 5A (B), which demonstrates that the utilization of visible light irradiation (in the conditions tested), also did not promote any changes to the chemical composition of treated enamel.

FIGS. 5A-B (C, I and O; HP [6%, 15% and 35%]), (F, L, and R; HP [6%, 15% and 35%]+LT), (D, J and P; HP [6%, 15% and 35%]+NP [5%]), (G, M and S; HP [6%, 15% and 35%]+NP [5%]+LT), (E, K and O; HP [6%, 15% and 35%]+NP [10%]) and (H, N and T; [6%, 15% and 35%]+NP [10%]+LT) illustrate the results for specimens that were subjected to experimental bleaching protocols. It is possible to observe that specimens treated with HP (either 6%, 15% and 35%) with or without LT (FIGS. 5A-B (C, F, I, L, O and R), displayed spectra at T₄ that were characterized by lower absorbance values for the CO₃² υ2 (886 cm⁻¹) and PO₄³ υ1 (996 cm⁻¹) peaks. In addition, it was possible to observe that the combination of HP and LT shifted the spectra to the right (wavenumbers between 800 cm⁻¹ and 1,150 cm⁻¹). Such behavior was more drastic for specimens treated with either 6% or 15% HP and LT (FIGS. 5A-B (F and L)). In combination, these results indicate that chemical composition of enamel has been altered by experimental bleaching protocols. This behavior was not observed on specimens treated with bleaching gels containing NF_TiO₂ independent of light irradiation (with or without) or nanoparticles' concentrations (either 5% or 10%). In these instances, spectra were observed to display shapes and absorbance values that were similar to those of the control group (no treatment, stored in artificial saliva) at T₄, thereby indicating that the presently disclosed NP-containing compositions can decrease potential adverse effects precipitating from dental bleaching procedures onto the chemical make-up of treated enamel.

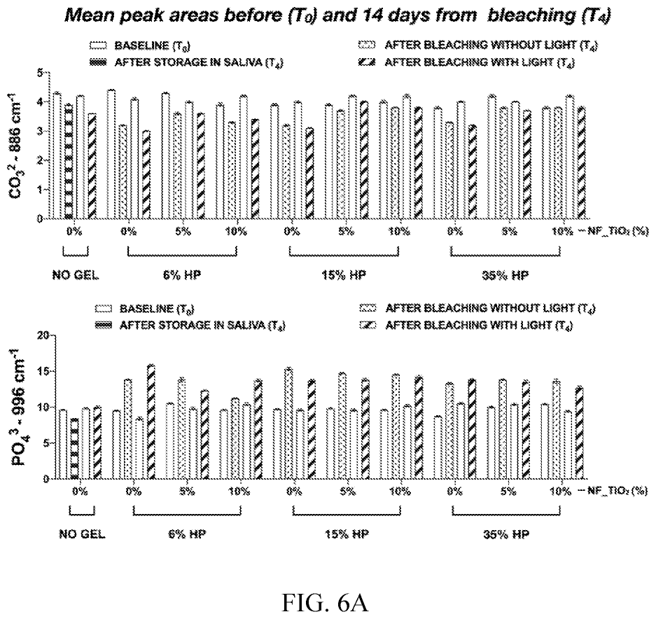

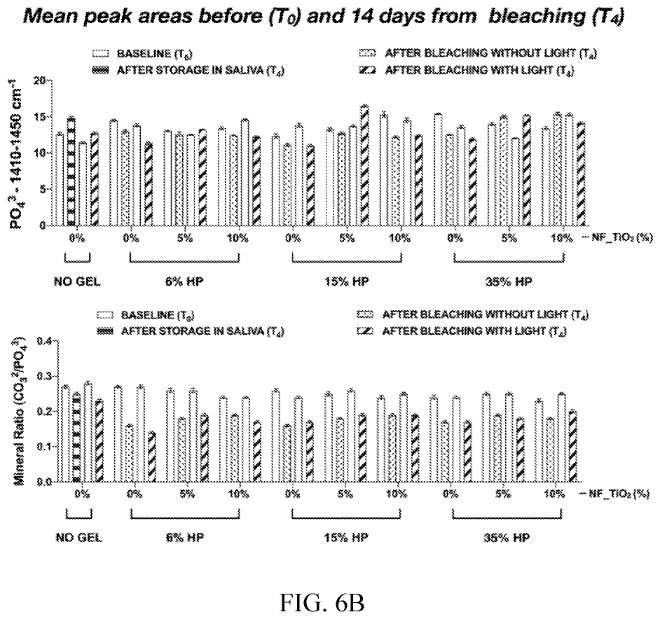

The results of the calculation of the areas under specific peaks (CO₃² υ2 [886 cm⁻¹], PO₄³ υ1 [996 cm⁻¹] and PO₄³ υ2 [1,410-1,460 cm⁻¹]) are shown in FIG. 6A (A-C). This approach was made necessary to allow the quantitatively characterization of the effects of experimental bleaching agents on the chemical composition of treated enamel and to determine the mineral ratio between carbonate and phosphate. It is clear from FIG. 6A (A), that, with the exception of specimens treated with 35HP+10NF_TiO₂ in dark conditions, lower concentrations of carbonate (CO₃² υ2) were observed at T₄ independently of the concentration of peroxide or nanoparticles investigated and values ranged from 3.0±0.02 (6HP+LT) to 3.9±0.03 (CONT). A similar behavior was also observed for specimens in G1 (negative control) and G2 (LT only). The smallest decrease in carbonate concentrations were observed in G11-G14 and G17-G20, which are experimental groups containing HP (either 15% or 35%) and NF_TiO₂ (either 5% or 10%) in both light irradiation conditions (with or without). In FIG. 6A (B), it becomes obvious that all experimental groups investigated were able to increase specimens' phosphate (PO₄³ υ1) concentrations at T₄ and values varied from 8.4±0.2 (CONT) to 15.8±0.1 (6% HP+LT).

FIG. 6A (C) demonstrate that experimental groups displayed levels of PO₄³ υ2 that were either comparable or higher to those in the negative control group, and values varied from 8.4±0.1 (CONT) and 15.8±0.5 (6HP+LT). FIG. 6A (D) illustrate the impact of experimental bleaching agents on the mineral ratio CO₃²/PO₄³ of bovine enamel. It is possible to observe a decrease at T₄ in all groups investigated where values varied from 0.14±0.03 (6HP+LT) to 0.20±0.05 (35HP+10NP+LT). In combination, these results indicate that specimens treated with experimental bleaching agents containing HP (either 6%, 15% or 35%) and NP (either 5% or 10%) displayed mineral content that was less altered when compared to specimens treated with bleaching gels without nanoparticles, thereby further indicating the ability of the disclosed technology to decrease potential adverse effects resulting from dental bleaching procedures.

### Topography Assessment

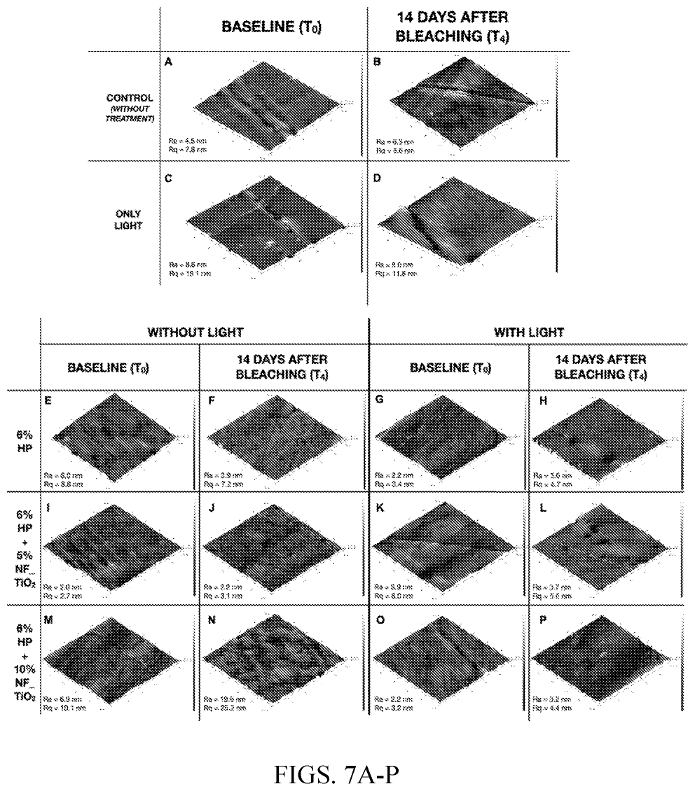

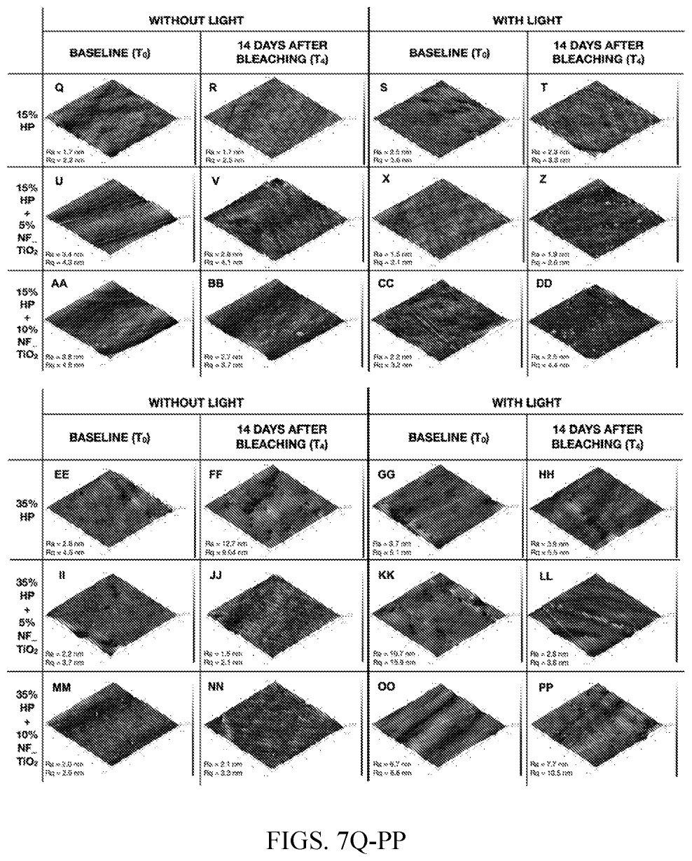

Illustrative results from the topographical assessment performed with AFM are shown in FIGS. 7A-B (A-PP) where it is possible to observe that Rₐ and R_{q} values varied from 1.5 nm (35HP+5NP at T₄) to 19.6 nm (6HP+10NP) and 2.1 nm (35HP+5NP) to 25.2 nm (6HP+10NP), respectively. Even though such large variations in Rₐ and R_{q} values could be correlated to intense damage of enamel, the vast majority of our results have indicated that treatments conducted with or without LT, and modulated by experimental gels containing (6%, 15% and 35%) with (5% and 10%) or without NP, were not able to adversely impact the surface topography of treated enamel at T₄, and therefore, results from the group treated with 6HP+10NP were considered not to be representative. These could have resulted from human error during polishing and finishing procedures or during AFM data collection. Results reported have also demonstrated that finishing and polishing procedures, described above, were not capable of completely removing gross irregularities produced during finishing steps, and were observed as straight lines of dark colors on multiple specimens. Results reported have also allowed us to observe the distribution and direction of enamel prisms (dragon scales in images FIG. 7A (B, I) in some specimens.

### Metabolic Status of Non-Disrupted Biofilms

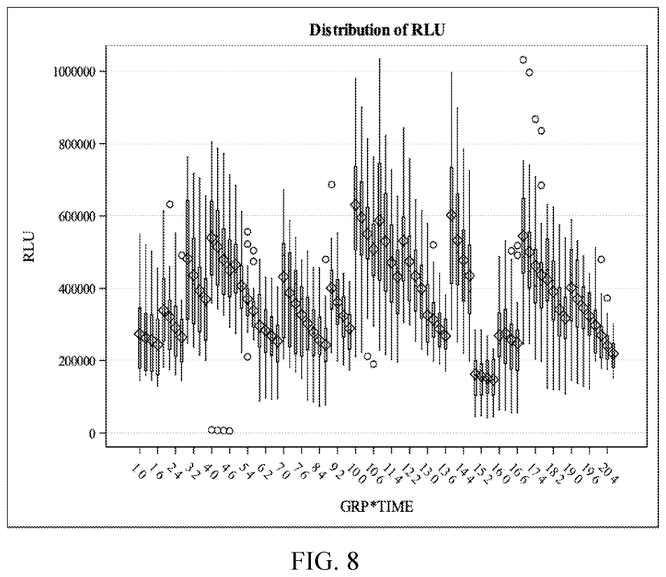

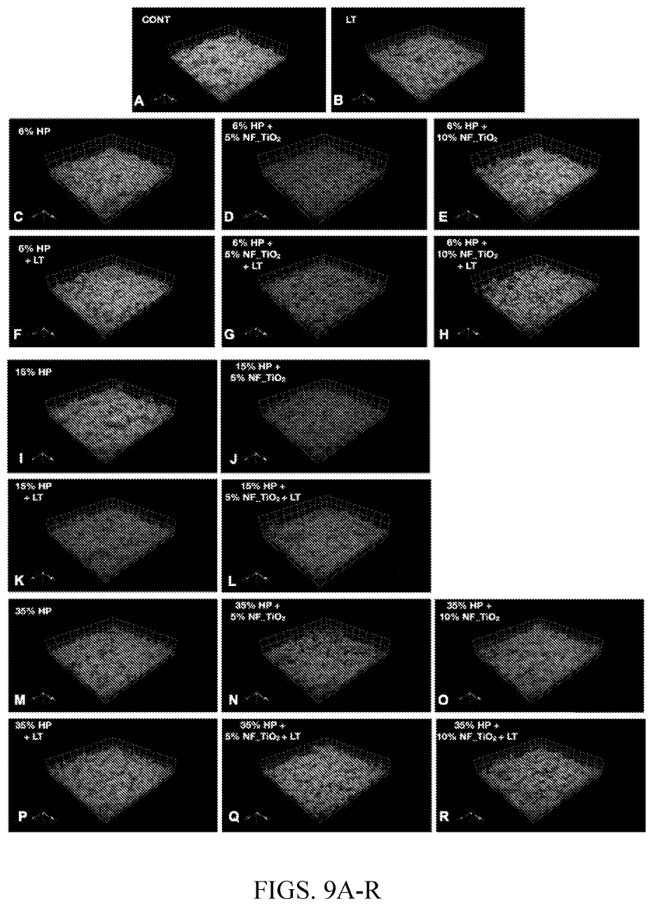

FIG. 8 illustrates the results (interaction between group and time), in terms of relative luminescence units (mean and standard deviation values; at 0, 2, 4 and 6 minutes after the addition of D-Luciferin), of the metabolic status of non-disrupted S. mutans biofilms grown (24 hours) on the surfaces of bleached enamel. It is possible to observe that, with the exception of G12 (HP6+10NP+LT) and G18 (HP6+10NP+LT), biofilms grown on specimens treated with experimental bleaching protocols modulated by gels containing HP (6%, 15% and 35%) with (5% and 10%) or without NP and LT, displayed metabolic statuses that were either comparable to or higher than those observed on G1 (negative control), which demonstrates that experimental bleaching protocols were not capable of displaying long-term antibacterial properties against S. mutans biofilms. The results from G15 and G16 indicate that biofilms were adversely impacted by the treatment, as denoted by RLU values that were smaller than those from G1 (negative control) and the establishment of a plateau-type of curve.

### Confocal Microscopy

The results from the concurrent staining and confocal microscopy analysis are show in FIG. 9 (A-R) as 3-D reconstructions of biofilms, where it is possible to observe in terms of green (nucleic acids), red (proteins) and blue (EPS) fluorescence, the impact of experimental bleaching treatments on components of biofilms and their 3-dimensional distributions. It becomes obvious from FIG. 9A, that biofilms expressed mostly green fluorescence when grown against the surfaces of specimens that were not treated with experimental bleaching gels (G1-negative control). Even though visible light irradiation (G2, 405±15 nm), in the conditions tested, was not expected to alter the surfaces of enamel (as shown by the AFM analysis), it was possible to observe that biofilms grown on light-irradiated surfaces were not only more porous, but also displayed fluorescence signals that were mostly red and blue, which indicates that biofilms were expressing proteins and EPS more intensely. A clear trend, in terms of fluorescence signals (either green, red or blue), could not be observed for specimens treated with experimental bleaching gels containing HP (6%, 15% or 35%) with (5% and 10%) or without NF_TiO₂ and LT, which were considered unexpected findings.

The presently disclosed results demonstrate that the incorporation of NPs (e.g., 5% and 10%, w/v) into experimental bleaching gels containing low concentrations of HP (e.g., 6% and 15%) rendered esthetic outcomes (in terms of ΔE_{ab}, ΔE₀₀ and ΔWI_{D}) that were similar to those attained with high-concentrated bleaching gels (HP35), thereby demonstrating that the disclosed compositions can resolve mild-to-severe dental discolorations in short periods of time (3 sessions, 30 min/session). The utilization of light irradiation (LT; 405 nm±15 nm) was shown to improve the efficacy of experimental bleaching gels containing varying concentrations of HP (6%, 15% and 35%) with or without NP (5% and 10%), as denoted by mean values of ΔE_{ab}, ΔE₀₀ and ΔWI_{D} that were higher than those from experimental bleaching gels (with or without NPs) in dark conditions, which demonstrates that LT is still fundamentally important to achieve good esthetic outcomes.

In summary, in at least certain embodiments, the present disclosure is directed to a dental bleaching composition that comprises a peroxide bleaching agent, a gel-forming thickening agent, and nitrogen-doped titanium dioxide nanoparticles, wherein the dental bleaching composition causes an increase in mineralization of the teeth to which it is applied without causing dentin hypersensitivity of the teeth. Optionally, the nitrogen-doped titanium dioxide nanoparticles of the dental composition may be co-doped with fluorine or silver, or both fluorine and silver. In the dental bleaching composition, the peroxide bleaching agent optionally may be selected from hydrogen peroxide (HP) and carbamide peroxide (CP). Further, in any of the above dental bleaching compositions, the peroxide bleaching agent may optionally be present in a weight to volume ratio in a range of 1% to 30%. Further, in any of the above dental bleaching compositions, the dental bleaching composition optionally may not comprise a dental adhesive material.

In at least certain embodiments, the present disclosure is directed to a method of whitening teeth that comprises applying to a subject's teeth a dental composition comprising a peroxide bleaching agent, a gel-forming thickening agent, and nitrogen-doped titanium dioxide nanoparticles, wherein the dental composition causes an increase in mineralization of the teeth to which it is applied without causing dentin hypersensitivity of the teeth. Optionally, the nitrogen-doped titanium dioxide nanoparticles of the dental composition may be co-doped with at least one of fluorine and silver. The method may comprise the step of applying the dental composition to a portion of the teeth which is not in need of dental repair. Further in any of the above methods, the peroxide bleaching agent of the dental composition optionally may be selected from hydrogen peroxide (HP) and carbamide peroxide (CP). In any of the above methods the peroxide bleaching agent may be present in a weight to volume ratio in a range of 1% to 30%. In any of the above methods, optionally the dental composition may be irradiated after it has been applied to the subject's teeth. In any of the above methods, optionally the dental composition may not comprise a dental adhesive material.

In at least certain embodiments, the present disclosure is directed to a method of enhancing mineralization in a subject's teeth, comprising applying to the subject's teeth a dental composition comprising a gel-forming thickening agent and nitrogen-doped titanium dioxide nanoparticles, wherein the dental composition causes an increase in mineralization of the teeth to which it is applied without causing dentin hypersensitivity of the teeth. Optionally, the nitrogen-doped titanium dioxide nanoparticles of the dental composition may be co-doped with at least one of fluorine and silver. Optionally any of the above the dental compositions may be applied to a portion of the teeth which is not in need of dental repair. Optionally, any of the above dental compositions may further comprise a peroxide bleaching agent. Optionally, any of the above peroxide bleaching agents may be selected from hydrogen peroxide (HP) and carbamide peroxide (CP). Optionally, any of the above peroxide bleaching agents may be present in a weight to volume ratio in a range of 1% to 30%. Optionally, any of the dental compositions may be irradiated after it has been applied to the subject's teeth. Optionally, any of the dental compositions described herein may not comprise a dental adhesive material.

1. A dental bleaching composition, comprising a peroxide bleaching agent, a gel-forming thickening agent, and nitrogen-doped titanium dioxide nanoparticles, and the dental bleaching composition causing an increase in mineralization of the teeth to which it is applied without causing dentin hypersensitivity of the teeth.

2. The dental bleaching composition of claim 1, wherein the peroxide bleaching agent is selected from hydrogen peroxide (HP) and carbamide peroxide (CP).

3. The dental bleaching composition of claim 1, wherein the peroxide bleaching agent is present in a weight to volume ratio in a range of 1% to 30%.

4. The dental bleaching composition of claim 1, wherein the dental bleaching composition does not comprise a dental adhesive material.

5. A method of whitening teeth, comprising applying to a subject's teeth a dental composition comprising a peroxide bleaching agent, a gel-forming thickening agent, and nitrogen-doped titanium dioxide nanoparticles, wherein the dental composition causes an increase in mineralization of the teeth to which it is applied without causing dentin hypersensitivity of the teeth.

6. The method of claim 5, comprising applying the dental composition to a portion of the teeth which is not in need of dental repair.

7. The method of claim 5, wherein the peroxide bleaching agent of the dental composition is selected from hydrogen peroxide (HP) and carbamide peroxide (CP).

8. The method of claim 5, wherein the peroxide bleaching agent of the dental composition is present in a weight to volume ratio in a range of 1% to 30%.

9. The method of claim 5, comprising irradiating the dental composition after it has been applied to the subject's teeth.

10. The method of claim 5, wherein the dental composition does not comprise a dental adhesive material.

11. A method of enhancing mineralization in a subject's teeth, comprising applying to the subject's teeth a dental composition comprising a gel-forming thickening agent and nitrogen-doped titanium dioxide nanoparticles, wherein the dental composition causes an increase in mineralization of the teeth to which it is applied without causing dentin hypersensitivity of the teeth.

12. The method of claim 11, comprising applying the dental composition to a portion of the teeth which is not in need of dental repair.

13. The method of claim 11, wherein the dental composition further comprises a peroxide bleaching agent.

14. The method of claim 13, wherein the peroxide bleaching agent of the dental composition is selected from hydrogen peroxide (HP) and carbamide peroxide (CP).

15. The method of claim 13, wherein the peroxide bleaching agent of the dental composition is present in a weight to volume ratio in a range of 1% to 30%.

16. The method of claim 11, comprising irradiating the dental composition after it has been applied to the subject's teeth.

17. The method of claim 11, wherein the dental composition does not comprise a dental adhesive material.

18. The dental bleaching composition of claim 1, wherein the nitrogen-doped titanium dioxide nanoparticles of the dental composition are co-doped with at least one of fluorine and silver.

19. The dental bleaching composition of claim 1, wherein the peroxide bleaching agent is present in a weight to volume ratio in a range of 6% to 12%.

20. The method of claim 5, wherein the nitrogen-doped titanium dioxide nanoparticles of the dental composition are co-doped with at least one of fluorine and silver.

21. The method of claim 5, wherein the peroxide bleaching agent of the dental composition is present in a weight to volume ratio in a range of 6% to 12%.

22. The method of claim 11, wherein the nitrogen-doped titanium dioxide nanoparticles of the dental composition are co-doped with at least one of fluorine and silver.

23. The method of claim 13, wherein the peroxide bleaching agent of the dental composition is present in a weight to volume ratio in a range of 6% to 12%.
